(19) United States
(12) Patent Application Publication
(10) Pub. No.: US 2022/0143159 A1
(43) Pub. Date: May 12, 2022

(54) Cystatin C and Cystatin 9 to Treat Inflammation Caused by Bacteria

(71) Applicant: Board of Regents, The University of Texas System, Austin, TX (US)

(72) Inventors: Tonyia Eaves-Pyles, Galveston, TX (US); Richard Pyles, Galveston, TX (US); Bernard P. Arulanandam, San Antonio, TX (US)

(21) Appl. No.: 17/550,098

(22) Filed: Dec. 14, 2021

Related U.S. Application Data
(62) Division of application No. 15/826,150, filed on Nov. 29, 2017, now abandoned.
(60) Provisional application No. 62/428,421, filed on Nov. 30, 2016.

(51) Int. Cl. A61K 38/57; A61K 38/12; A61K 47/60; A61K 45/06; A61P 31/04; A61P 31/00; A61P 29/00

(52) U.S. Cl. CPC A61K 38/57; A61K 38/12; A61K 47/60; Y02A 50/30; A61P 31/04; A61P 31/00; A61P 29/00; A61K 45/06

(57) ABSTRACT

The present invention includes a composition and method for controlling an immune response in a host to a pathogenic bacterial infection comprising: identifying a subject in need of treatment for infection with a pathogenic bacteria; and providing a composition comprising recombinant Cystatin 9 (CST9), a cystatin C (CSTC), or both CST9 and CSTC, in an amount sufficient to restrain or prevent a life-threatening, unrestrained systemic inflammatory response syndrome in a host against a pathogenic bacteria.

### CROSS-REFERENCE TO RELATED APPLICATIONS

This application is a Divisional application of U.S. patent application Ser. No. 15/826,150, filed on Nov. 29, 2017 and claims priority to U.S. Provisional Patent Application Ser. No. 62/428,421, filed on Nov. 30, 2016, entitled “Cystatin C and Cystatin 9 to Treat Inflammation Caused by Bacteria” the contents of which is incorporated by reference herein in its entirety.

### STATEMENT OF FEDERALLY FUNDED RESEARCH

This invention was made with government support under R21A106877402 awarded by NIH/NIAID. The government has certain rights in the invention.

### TECHNICAL FIELD OF THE INVENTION

The present invention relates in general to the field of inflammation, and more particularly, to treatment of conditions such as sepsis and septic shock.

### BACKGROUND OF THE INVENTION

Without limiting the scope of the invention, its background is described in connection with systemic inflammatory conditions.

Systemic inflammatory conditions are one class of diseases for which early diagnosis is particularly desirable, with sepsis being the most serious. Sepsis is the result of the interaction of a pathogenic microorganism with a host's immune system that leads to systemic inflammation. The characterization of sepsis in a host is very complex due to a heterogeneity of factors that play into the final outcome. A number of factors are drivers of the underlying immune response and systemic inflammatory disease, such as, a patient's genetically determined response to immune stimuli, the general status of their immune system, and microbial mediators and virulence factors released by infectious organisms. The progression of a systemic inflammatory diseases is often remarkably rapid, leaving the clinician with little time to make a considered clinical judgment.

U.S. Pat. No. 9,351,983, issued to Corda, et al., entitled, “Use of glycerophosphoinositols for the treatment of septic shock” teaches the use of glycerophosphoinositols (GPIs) and derivatives thereof for use in the treatment of pathologies related to a Lipopolysaccharide (LPS)-activated tissue-factor (TF) activity, as pathologies induced by high bacteremia, i.e. septic shock.

U.S. Pat. No. 9,227,997, issued to Park, et al., entitled, “Composition for treating sepsis or septic shock comprising the peptide originated from the Smad6” teaches a pharmaceutical composition comprising a Smad6-derived peptide as an active ingredient. This composition is said to have the ability to specifically bind to Pellino-1, the Smad6-derived peptide is effectively useful in the treatment of the sepsis mediated by excessively activated TLR, and effectively reduce the expression of inflammatory cytokines, protects cells from sepsis-induced apoptosis, and exhibits high bacterial clearance in animal models of sepsis.

U.S. Pat. No. 7,022,734, issued to Kazmierski, et al., entitled, “Treatment of septic shock” teaches the use of transition metal complexes in the treatment of septic shock, in particular, the hypotension associated therewith and pharmaceutical formulations comprising such complexes are disclosed. The use of such transition metal complexes in the treatment of other conditions caused by pathological NO production are also said to be disclosed.

### SUMMARY OF THE INVENTION

In one embodiment, the present invention includes a composition comprising: a recombinant Cystatin 9 (CST9), Cystatin C (CSTC), or both in an amount sufficient to restrain or prevent a life-threatening, unrestrained systemic inflammatory response syndrome in a host against a pathogenic bacteria. In one aspect, the pathogenic bacteria is selected from at least one of: Burkholderia thialandensis, Klebsellia pneumoniae, E. coli O157:H7, Pseudomonas aeruginosa, or Salmonella typhimurium. In another aspect, the pathogenic bacteria is multiple drug resistant. In another aspect, the pathogenic bacteria is not Francisella tularensis or an obligate intracellular pathogen. In another aspect, the composition is concurrently administered with one or more antibiotics that are bacteriocidal or bacteriostatic against the pathogenic bacteria. In another aspect, the composition further comprises one or more antibiotics that are bacteriocidal or bacteriostatic against the pathogenic bacteria. In another aspect, the composition is adapted for controlled release over a 4, 6, 8, 12, or 14 hour period. In another aspect, the pathogenic bacteria are Gram negative bacteria. In another aspect, the composition is adapted for intraperitoneal, intravenous, parenteral, enteral, pulmonary, intranasal, intramuscular, rectal, or oral administration. In another aspect, both Cystatin 9 (CST9) and Cystatin C (CSTC) are provided concomitantly. In another aspect, the CST9 and the CSTC are provided in a synergistic amount. In another aspect, the Cystatin 9 (CST9) and Cystatin C (CSTC) are provided in an amount of 1-500 picograms/kilo. In another aspect, the composition further comprises a synergistic amount of a polymyxin antibiotic. In another aspect, the composition further comprises a sub-optimal dose of a polymyxin antibiotic, wherein the dose is not neurotoxic, nephrotoxic, or both. In another aspect, the composition further comprises a synergistic amount of colistin.

In another embodiment, the present invention includes a method of controlling an immune response in a host to a pathogenic bacterial infection comprising: identifying a subject in need of treatment for infection with a pathogenic bacteria; and providing a composition comprising recombinant Cystatin 9 (CST9), a cystatin C (CSTC), or both CST9 and CSTC, in an amount sufficient to restrain or prevent a life-threatening, unrestrained systemic inflammatory response syndrome in a host against a pathogenic bacteria. In one aspect, the systemic inflammatory response syndrome is an acute lung injury or an acute respiratory distress syndrome. In another aspect, the systemic inflammatory response syndrome is septic shock. In another aspect, the composition is provided concurrently with one or more antibiotics that are bacteriocidal or bacteriostatic against the pathogenic bacteria. In another aspect, the composition further comprises one or more antibiotics that are bacteriocidal or bacteriostatic against the pathogenic bacteria. In another aspect, the composition is adapted for controlled release over a 4, 6, 8, 12, or 14 hour period. In another aspect, the pathogenic bacteria is selected from at least one of: Burkholderia thialandensis, Klebsellia pneumoniae, E. coli O157:H7, Pseudomonas aeruginosa, or Salmonella typhimurium. In another aspect, the pathogenic bacteria is multiple drug resistant. In another aspect, the pathogenic bacteria is not Francisella tularensis or an obligate intracellular pathogen. In another aspect, the pathogenic bacteria is Gram negative. In another aspect, the composition is adapted for intraperitoneal, intravenous, parenteral, enteral, pulmonary, intranasal, intramuscular, rectal, or oral administration. In another aspect, the CST9 and the CSTC are provided in a synergistic amount. In another aspect, both Cystatin 9 (CST9) and Cystatin C (CSTC) are provided intranasally when the systemic inflammatory response syndrome is an acute lung injury, an acute respiratory distress syndrome, or both. In another aspect, the Cystatin 9 (CST9) and Cystatin C (CSTC) are provided in an amount of 1-500 picograms/kilo. In another aspect, the composition further comprises a synergistic amount of a polymyxin antibiotic. In another aspect, the composition further comprises a sub-optimal dose of a polymyxin antibiotic, wherein the dose is not neurotoxic, nephrotoxic, or both. In another aspect, the composition further comprises a synergistic amount of colistin.

In another embodiment, the present invention includes a method of controlling an immune response in a host to a pathogenic bacterial infection comprising: identifying a subject in need of treatment for infection with a pathogenic bacteria; and administering a composition comprising recombinant Cystatin 9 (CST9), a cystatin C (CSTC), or both CST9 and CSTC, in an amount sufficient to restrain or prevent a life-threatening, unrestrained systemic inflammatory response syndrome in a host against a pathogenic bacteria. In one aspect, the step of administering the recombinant Cystatin 9 (CST9), a cystatin C (CSTC), or both CST9 and CSTC is at least one of: after the onset of symptoms, at least three days post-infection, or at least three days post-exposure. In another aspect, the CST9 and the CSTC are provided in a synergistic amount. In another aspect, the composition further comprises a synergistic amount of a polymyxin antibiotic. In another aspect, the composition further comprises a sub-optimal dose of a polymyxin antibiotic, wherein the dose is not neurotoxic, nephrotoxic, or both. In another aspect, the composition further comprises a synergistic amount of colistin.

### BRIEF DESCRIPTION OF THE DRAWINGS

For a more complete understanding of the features and advantages of the present invention, reference is now made to the detailed description of the invention along with the accompanying figures and in which:

FIG. 1 shows the establishment of LD90 NDM-1 Kp pneumonia murine model. Balb/c mice (n=20 mice/group) were intranasally (i.n.) inoculated with various challenge doses of NDM-1 Kp and their survival was observed for 10d. The LD90 of Kp resulted in 1.82×10⁸ CFU per mouse.

FIG. 2 shows an example of optimal rCST9/rCSTC treatment regimens affords protection against NDM-1 Kp pneumonia. Balb/c mice (n=20 mice/gp) were i.n. infected with NDM-1 Kp (1.82×10⁸ CFU/mouse) and then treated as follows: mice were given an i.n. dose of rCST9/rCSTC (50 pg of each/mouse) at 1 h PI followed by 500 pg of both rCST9/rCSTC/mouse on the 3d PI or mice were administered a single i.p. dose of rCST9/rCSTC (500 pg of each/mouse). Both rCST treatment regimens significantly increased survival compared to that in NDM-1 Kp-infected mice alone (p<0.05). Data are presented as mean±SEM, and the asterisk signifies significant differences of p<0.05.

FIG. 3 shows the results from pegylation of rCST9, co-administered with rCSTC, did not improve survival outcomes of NDM-1 Kp infected mice. Balb/c mice (n=15 mice/gp) were i.n. infected with an LD90 challenge with NDM-1 Kp and then treated with an i.n. dose of PEG-rCST9/rCSTC (50 pg of each/mouse) on 1 h PI followed by 500 pg of PEG-rCST9/rCSTC/mouse at 3d PI or mice were administered a single i.p. dose of PEG-rCST9/rCSTC (500 pg of each/mouse) at 3d PI. PEG-rCST9/rCSTC did not improve the survival of infected mice that surpassed rCST9/rCSTC. However, both groups that receive the i.p. administration of rCST9 or PEG-rCST9 in combination with rCSTC on day 3 PI significantly increased survival compared to NDM-1 Kp infected mice (p<0.05). Data are presented as mean±SEM, and asterisk signifies significant differences of p<0.05.

FIG. 4 shows that rCST9/rCSC treatment improves survival alone or in combination with a suboptimal low dose of colistin in mice infected with a lethal pulmonary challenge with NDM-1 Kp. Balb/c mice (n=15 mice/gp) were i.n. infected with NDM-1 Kp (1.82×10⁸ CFU/mouse) and then given an i.p. dose of 500 pg of both rCST9/rCSTC/mouse at 3d PI. Then mice were given colistin at 20 or 1.25 mg/kg/mouse i.p. 2 times/day for 2 days at 4 and 5d PI. rCST treatment worked synergistically with a very low dose of colistin (1.25 mg/kg) to significantly improve and show synergistic survival compared to infected mice (p<0.05), and 1.25 mg/kg of colistin alone. Therefore, rCSTs significantly extended the period of survival before suboptimal low dose of antibiotic treatment was needed/initiated. Data are presented as mean±SEM, and asterisk signifies significant differences of p<0.05.

FIG. 5A shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Serum was collected and lungs, livers, and spleens were harvested at 5 days PI. Fold change in the overall cytokine levels in the serum (FIG. 5A) of rCST treated mice were decreased compared to untreated in NDM-1 infected mice.

FIG. 5B shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Serum was collected and lungs, livers, and spleens were harvested at 5 days PI. Fold change in the overall cytokine levels in the lungs (FIG. 5B) of rCST treated mice were decreased compared to untreated in NDM-1 infected mice.

FIG. 5C shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments modulated cytokine secretion in the serum (FIG. 5C).

FIG. 5D shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments modulated cytokine secretion in all tested organs (FIG. 5D).

FIGS. 5E to 5H shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments significantly reduced bacterial burden in the lungs (E). Lung histology (H&E; 40×mag) from the same treated and/or infected mice showed that both rCST treatment regimens minimalized lung pathology caused by NDM-1 Kp (FIG. 5F). rCST treatment reduced apoptotic cells compared to untreated/infected mice (FIG. 5G). MDA detection in the lungs was significantly decreased in rCST-treated and infected mice (FIG. 5H). Data are presented as mean±SEM, and asterisk signifies significant differences of p<0.05.

FIGS. 6A to 6C show that rCST treatments preserved lung integrity and prevented long-term lung damage. Balb/c mice (n=4 mice/gp) were i.n. infected with NDM-1 Kp (1.82×10⁸ CFU/mouse) and then treated with an i.n. dose of rCST9/rCSTC (50 pg of each). The lungs were harvested at 24 h and 72 h PI. A parallel group of mice were infected and treated i.n./i.p. or i.p with rCST9/rCSTC as described herein, and then lungs were harvested on 5 and 10d PI. Serial sections of the lung were analyzed for histology (40×mag) and apoptosis by using the TUNEL assay with DAPI to stain cell nuclei. FIG. 6A shows the i.n. administration of rCST9/rCSTC to infected mice markedly diminished immune cell infiltration into the lungs and edema at 24 h and 72 h PI compared to high cellularity and signs of hemorrhaging and edema in the lungs of untreated, infected mice. Further, lungs from our two optimal rCST9/rCSTC treatments on 5d and 10d PI prevented long-term lung damage and showed resolution of inflammation. FIG. 6B shows histolopathological scoring of the lungs (0=no significant changes, 1=slight damage, 2=mild to moderate damage, 3=moderate to severe damage and 4=severe damage in each of the three categories. Results showed that cystain treatments significantly decreased lung damage compared to corresponding time points of infected mice alone. Mice receiving rCSTs at 3d PI and lungs collected from survivors at 5 and 10d PI had mild to no damage compared to infected mice alone groups (*p<0.05 and **p<0.01 respectively). The scoring results were expressed as SQS (mean±SEM). FIG. 6C shows likewise, lungs from the same rCST-treated and infected groups showed markedly fewer apoptotic cells at 24 and 72 h PI. All images are representative of the analysis of 4-6 sections of each mouse.

FIGS. 7A to 7C show the anti-microbial properties of rCST9/rCSTC against NDM-1 Kp. rCST9/rCSTC inhibited the metabolic activity and growth of NDM-1 Kp. The 50, 500, and 1000 pg of rCST9/rCSTC decreased metabolic activity (FIG. 7A), bacterial replication (FIG. 7B) and growth (FIG. 7C) NDM-1 Kp (1×10⁶ CFU/mL) following a 6 h incubation. Data are presented as mean±SEM, and asterisk signifies significant differences of p<0.05 compared to all other groups.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Cystatins are inhibitors of the lysosomal cysteine proteinases, cathepsin B, L, H and S and can function extracellularly and intracellularly. These inhibitors have been shown to regulate and give protection to the host against uncontrolled proteolysis in various disease processes namely cancer and neurodegeneration. As such cystatins have been shown to modulate inflammation, prevent the metastasis of tumor cells and protect healthy tissue against penetration of bacteria. Therefore, cystatins have been studied as a potential therapeutic agent for certain cancers and a biomarker for antitumor therapy. Additionally, cystatin was upregulated in LP-stimulated cancer cells suggesting that the inhibition of the secreted form of cysteine proteinases prevents inflammatory tissue injury. Cystatin 9 (CST9) and cystatin C (CSTC) are a small 18 kDa protein that can be secreted and found in human fluids as well as expressed in the lungs, liver, heart, pancreas, skeletal muscle and placenta. Although little is known about CST9, it is thought to play a role in inflammation but it's role is unknown specifically during pathogenic bacterial infections. CSTC has been well studied as a potential therapeutic agent to prevent and/or restrain neurodegeneration and metastasis of tumor cells. Cystatin 9 (Homo sapiens cystatin 9 (testatin) (CST9) has NCBI Reference Sequence: NM 001008693.2, Gene ID: 128822, relevant sequences incorporated herein by reference. Cystatin C (CSTC) or Homo sapiens cystatin C (CST3), transcript variant 2, mRNA, has NCBI Reference Sequence: NM 001288614.1, Gene ID: 1471, relevant sequences incorporated herein by reference.

As used herein, the term “pharmaceutically effective amount” refers to that amount of an agent effective to produce the intended effect of reducing, preventing and/or modulating immune responses and thereby inducing controlled, beneficial inflammation against bacterial pathogens, such as Gram negative bacteria or multiple drug resistant bacteria. Generally, the compositions and method of the present invention prevent or reduce run-away immune responses caused by an over-reaction by the immune response to the pathogen, leading to severe immune-mediated shock, e.g., septic shock, acute lung injury (ALI), acute respiratory distress syndrome (ARDS), and systemic inflammation. Such factors include the generation of a cytokine cascade, hypercytokemia, or cytokine storm that is a potentially fatal immune reaction. For example, the cytokine storm generally includes a positive feedback loop between cytokines and white blood cells leading to highly elevated levels of various cytokines. It has been found that the present invention helps to ameliorate or lessen the release of both pro-inflammatory cytokines (e.g., Tumor Necrosis Factor-alpha, Interleukin-1, and Interleukin-6) and anti-inflammatory cytokines (e.g., Interleukin-10 and Interleukin-1 receptor antagonist), which are commonly elevated in the serum of patients experiencing a cytokine storm. Specifically, but not a limitation of the present invention, the present inventors have studied the effect of the compositions and methods of the present invention looking at, at least, GRO-alpha/KC/CINC1, IL-1Beta, MIP-lalpha, TNF-alpha, IL-6, IP-10 and IL-23.

As used herein, the term “cytokine storm” refers to the dysregulation of cytokines leading to disease that is also referred to as “cytokine release syndrome” or “inflammatory cascade”. Often, a cytokine storm or cascade is referred to as being part of a sequence because one cytokine typically leads to the production of multiple other cytokines that can reinforce and amplify the immune response. Generally, these pro-inflammatory mediators have been divided into two subgroups: early mediators and late mediators. Early mediators, such as e.g., Tumor-Necrosis Factor, Interleukin-1, Interleukin-6, are not sufficient therapeutic targets for re-establishing homeostatic balance because they are resolved within the time frame of a patient's travel to a clinic to receive medical attention. In contrast, the so-called “late mediators” have been targeted because it is during this later “inflammatory cascade” that the patient realizes that he or she has fallen ill.

In certain aspects the Cystatin 9 or Cystatin C is postranslationally modified by changes in, e.g., glycosylation, lipidation, PEGylation, and the like, to enhance one or more physiological characteristics during use, such as, e.g., increased resistance to degradation, increased half-life, enhanced activity, etc.

Generally, a cytokine cascade is a healthy systemic expression of the immune system, however, when the cascade enters a positive feedback loop without control it is referred to as a cytokine storm. The present invention can be used to reduce or eliminate some or most of an exaggerated immune response caused by, e.g., rapidly proliferating and highly activated T-cells or natural killer (NK) cells that results in the release of the “cytokine storm” that can include more than 150 inflammatory mediators (cytokines, oxygen free radicals, and coagulation factors). Both pro-inflammatory cytokines (such as Tumor Necrosis Factor-α, Interleukin-1, and Interkeukin-6) and anti-inflammatory cytokines (such as Interleukin-10, and Interleukin-1 receptor antagonist (IL-1RA)) become greatly elevated in, e.g., serum. It is this excessive release of inflammatory mediators that triggers the “cytokine storm.”

In the absence of prompt intervention, such as that provided by the present invention, a cytokine storm can result in permanent lung damage and, in many cases, death. The end stage symptoms of the cytokine storm include but are not limited to: hypotension; tachycardia; dyspnea; fever; ischemia or insufficient tissue perfusion; uncontrollable hemorrhage; severe metabolism dysregulation; and multisystem organ failure.

As used herein, the term “treatment” refers to the treatment of the conditions mentioned herein, particularly in a patient who demonstrates symptoms of the disease or disorder. As used herein, the term “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing, treating, eradicating, ameliorating or otherwise reducing the severity of the condition being controlled.

As used herein, the terms “effective amount” or “therapeutically effective amount” refer to an amount of a subject compound that will elicit the biological or medical response of a tissue, system, animal or human that is being sought by the researcher, veterinarian, medical doctor or other clinician. In one example, the therapeutically effective amount comprises 1 to 500 picograms/kg, 10 to 100 picograms/kg, 25 to 75 picograms/kg, 10, 20, 30, 40, 50, 60, 70, 80, 90, or 100, 125, 150, 175, 200, 225, 250, 275, 300, 325, 350, 375, 400, 425, 450, 475 and 500 picograms/kg of body weight of the subject.

As used herein, the terms “administration of” or “administering a” when referring to a compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as intravenous (IV), intramuscular (IM), or intraperitoneal (IP), intranasal (IN), intrapulmonary, and the like; enteral or parenteral, transdermal dosage forms, including creams, jellies, powders, or patches; buccal dosage forms; inhalation powders, sprays, suspensions, and the like; and rectal suppositories. For example, the term “intravenous administration” includes injection and other modes of intravenous administration, and likewise for the other routes of administration.

As used herein, the term “pharmaceutically acceptable” describes a carrier, diluent or excipient must be compatible with the other ingredients of the formulation and not deleterious to the recipient thereof.

As used herein, the term “systemic inflammatory response syndrome (SIRS)” refers to a clinical response to a variety of severe clinical insults, as manifested by two or more of the following: heart rate (HR)>90 beats/minute; respiratory rate (RR)>20 breaths/minute; P_{CO2}<32 mmHg, or requiring mechanical ventilation; temperature >38° C. or <36° C.; white blood cell count (WBC) either >12×10⁹/L or <4.0×0⁹/L or having >10% immature forms (bands), generally, within a 24 hour period. It is recognized that this represents a consensus definition of SIRS, and that the definition may be modified or supplanted by an improved definition in the future. The present definition is used herein to clarify current clinical practice, and does not represent a critical aspect of the invention.

The compositions of the present invention are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the composition may be formulated to provide, e.g., maximum and/or consistent dosing for the particular form for oral, rectal, topical, intravenous injection or parenteral administration. While the composition may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); Remington: The Science and Practice of Pharmacy, Pharmaceutical Press; 22nd Edition (2012); all of which are incorporated by reference, and the like, relevant portions incorporated herein by reference.

Although certain levels of inflammation are required for protection against infection, unrestrained inflammation can worsen an infection and/or disease. In fact, exacerbated inflammation can be an advantage to invasion by some mucosal pathogens. Current anti-inflammatory agents typically suppress/inhibit immune responses and/or fail to modulate the extent and intensity of the inflammatory cascade, compromising many aspects of acquired immunity development and leading to an imbalance of immune functions and increased risk of exacerbated primary and/or secondary infections. The present invention includes the immunomodulatory functions of the recombinant human protein rCST9 and rCSTC as an innovative and effective approach to modulate immune responses and thereby inducing controlled, beneficial inflammation against Gram-negative pathogens. rCST9 and rCSTC also generated direct antimicrobial outcomes that contributed to significant improvements in survival outcomes in several challenge models.

The present inventors show herein that rCST9 and rCSTC restrain and/or prevent life-threatening, unrestrained immune responses in the host against pathogenic bacteria. They further show that rCST9 and rCSTC have protective effects against various bacterial pathogens thus they could be potentially used as a treatment (alone and/or in combination with other treatments such as antibiotics) and/or intervention for multiple infectious agents.

Following cloning and purification of rCST9, the inventors show the protective effects engendered by rCST9 (50 pg dose) whereby this inhibitor retarded the viability and virulence of various pathogenic bacterial strains such as Burkholderia thialandensis (ATCC), Klebsellia pneumoniae (isolate from burn patient lung), E. coli O157:H7 (ATCC), Pseudomonas aeruginosa (isolate from burn patient wound), and Salmonella typhimurium (ATCC). Further, rCST9 induced human monocyte-derived macrophage (MDM) killing of these same pathogens. Additionally, both rCST9 and rCSTC, given individually and in combination with one another, inhibited the replication of the multi-drug resistant New Delhi metallo-β-lactamase (NDM-1)-producing Klebsellia pneumonia (Kp). Likewise, rCSTC (50 pg) and rCST9 (25 or 50 pg), given individually and in combination, restrained cytokine secretion from monocyte-derived macrophages (MDM) infected with New Delhi metallo-β-lactamase (NDM-1)-producing Kp compared to Kp-infected MDM. These in vivo data show that the combination of rCST9 and rCSTC administered at a dose of 50 pg/CST intranasally (i.n.) and/or 500 pg intraperitoneally (i.p.) significantly improved host survival in an experimental model of pneumonia caused by drug-resistant Delhi metallo-beta-lactamase-1 (NDM-1) producing Kp.

It is known that intestinal leakage of intestinal bacteria and their product occurs following burn injury likely contributing to systemic infection and sepsis. The present inventors have shown that individual treatments of rCST9 or rCSTC or the combination of both decreased IL-8 secretion from intestinal epithelial cells following exposure to enteric E. coli O83:H1 and/or flagellin. Balb/c mice gavaged with rCST9 or rCSTC (50 pg/mouse) then immediately given a 30% full-thickness burn showed a decrease in systemic MIP-1 and IL-8 secretion, specifically in the lungs, as induced by gut-derived enteric E. coli O83:H1 and/or flagellin as compared to controls. Additionally, rCST9 and rCSTC preserved the gut microbiome in these same animals.

The optimized recombinant cystatin 9 (rCST9) or PEG recombinant cystatin C (rCST9) were used to minimize Acute lung injury/acute respiratory distress syndrome (ALI/ARDS) and systemic inflammation resulting in improved survival (e.g. increased survival of 10-25% vs. infection alone) in an experimental mouse model of drug-resistant bacterial and viral pneumonia. The most effective dosage regime for rCST9 against New Delhi metallo-beta-lactamase-1 (NDM-1)-producing K. pneumoniae (Kp) pneumonia can be determined.

To establish the optimal CST route, timing, and dosage regimen against MDR NDM-1 Kp-induced pneumonia, Balb/c mice (n=10-20 mice/gp) were infected with an i.n. LD90 and treated at various times and doses as depicted in Table 1. Doses included i.n. and/or i.p. single rCST9, rCSTC or a combination of rCST9 and rCSTC. Mice that were infected with NDM-1 Kp but were not treated served as controls. rCST treatments by routes, doses, or as a combined therapy were studied to select an optimal regimen. The results showed that the i.n. monotherapy treatment with either rCST9 or rCSTC at 50 or 500 pg delivered 1d and/or 3d PI increased survival between 5-20% compared to findings with untreated NDM-1 Kp-infected mice. However, specific timing and route of the combination of rCST9/rCSTC PI markedly improved survival of Kp-infected mice compared to those given individual treatments (Table 1). Mice treated i.n. with rCST9/rCSTC (50 pg of each) at 1 h PI and then given rCST9/rCSTC (500 pg of each) i.p. at 3d PI significantly improved survival outcomes compared to the other groups in the study except one additional dosage regimen (Table 1; p<0.05). Interestingly, nearly equivalent survival was observed when a single i.p. dose of rCST9/rCSTC (500 pg/mouse) was given 3d PI compared to survival rates in untreated NDM-1 Kp-infected mice (Table 1; <0.05).

Evaluate optimal timing and dosage of rCST-9 and/or PEG-rCST9 administration post-infection. Balb/c mice (n=10-20 mice/gp) were intranasally (i.n.) infected with approximately 1.82×108 CFU/mouse of Kp 2146 (as established hereinabove) then treated i.n. or i.p. with rCST9, human recombinant cystatin C (rCSTC) or a combination of rCST9 and rCSTC as depicted in Table 1. Kp infected alone served as controls. Additionally, another cysteine proteinase inhibitor, CSTC, has been shown to induce anti-apoptotic pathways in neurons and possess anti-microbial activity against Gram-positive pathogens. Therefore, as a comparative control to rCST9, the inventors evaluated the effectiveness of rCSTC against pneumonia using the same doses of 50 and/or 500 pg/mouse. These results show that individually administered rCST9 or rCSTC increased survival 10-15% of Kp-infected mice (LD90) depending on timing (1 h and or/3d post-infection [PI]), route (i.n. and/or intraperitoneal (i.p.)) and dose (50 pg and/or 500 pg) [Table 1]. Table 1 shows the results from Cystatin treatment regime(s) against the multi-drug resistant NDM-1 Kp in pneumonia murine.

TABLE 1
Cystatin treatment regimens to combat NDM-1 Kp in pneumonia.

| | Dose per | | Administration | Administration | Increased % survival |
|---|---|---|---|---|---|
| Treatment | mouse | Route | Pre-infection | Post-infection | of Kp LD90 mice |
| rCST9 or rCSTC | 50 pg | i.n. | 1 h | | 10%, 20% respectively |
| rCST9 or rCSTC | 500 pg | i.n. | 1 h | | 10% |
| rCST9 or rCSTC | 50 pg | i.n. | | 3 d | 10% |
| rCST9 or rCSTC | 500 pg | i.n. | | 1 h and 3 d | 10% |
| rCST9 or rCSTC | 50 pg | i.p. then i.p. | | 1 h and 3 d | 5% |
| rCST9 and rCSTC | 50 pg, 500 pg | i.n. then i.p. | | 1 d and 3 d | 20% |
| rCST9 and rCSTC | 500 pg | i.p. then i.n. | | 1 d and 3 d | 10% |
| rCST9 and rCSTC | 500 pg | i.p. | | 4 d | 5% |
| rCST9 and rCSTC | 500 pg | i.p. | | 5 d | 0% |
| rCST9 and rCSTC | 500 pg | i.p. | | 1 d and 3 d | 5% |
| rCST9 and rCSTC | 50 pg, 500 pg | i.n. then i.p. | | 1 d and 3 d | 25% |
| rCST9 and rCSTC | 50 pg, 500 pg | i.n. then i.p. | | 1 h and 3 d | 38% |
| rCST9 and rCSTC | 500 pg | i.p. | | 3 d | 35% |

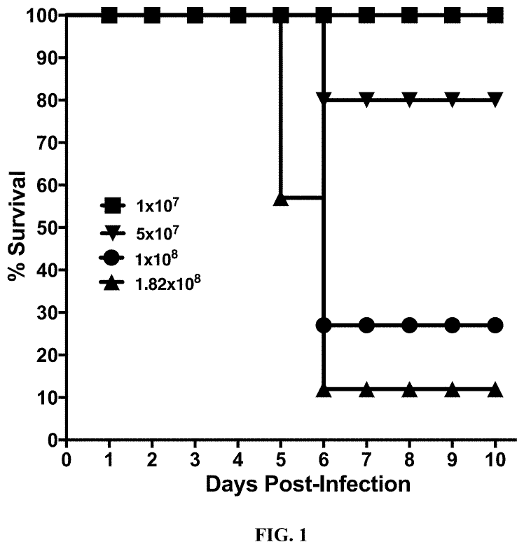

The inventors observed comparable survival rates when Kp infected mice were treated with individual rCST9 or rCSTC. The single i.p. dose of rCST9 or rCSTC at 50 pg/mouse increased survival of Kp infected mice by 15% [Table 1]. However, the combination of rCST9 and rCSTC (rCST9/rCSTC) markedly improved survival compared to individual treatments [Table 1; italicized groups]. FIG. 1 shows the establishment of LD90 NDM-1 Kp pneumonia murine model. Balb/c mice (n=20 mice/group) were intranasally (i.n.) inoculated with various challenge doses of NDM-1 Kp and their survival was observed for 10d. The LD90 of Kp resulted in 1.82×10⁸ CFU per mouse.

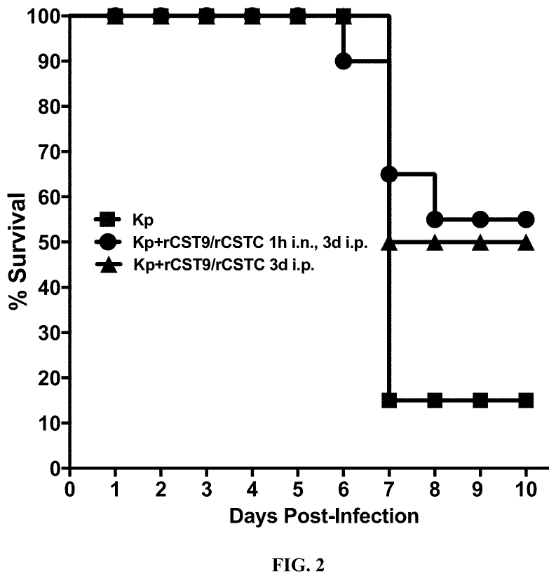

FIG. 2 shows an example of optimal rCST9/rCSTC treatment regimens affords protection against NDM-1 Kp pneumonia. Balb/c mice (n=20 mice/gp) were i.n. infected with NDM-1 Kp (1.82×10⁸ CFU/mouse) and then treated as follows: mice were given an i.n. dose of rCST9/rCSTC (50 pg of each/mouse) at 1 h PI followed by 500 pg of both rCST9/rCSTC/mouse on the 3d PI or mice were administered a single i.p. dose of rCST9/rCSTC (500 pg of each/mouse). Both rCST treatment regimens significantly increased survival compared to that in NDM-1 Kp-infected mice alone (p<0.05). Data are presented as mean±SEM, and the asterisk signifies significant differences of p<0.05.

Optimization of PEGylation rCST9: The PEGlyation protocol can be optimized and tested on BSA and are currently PEGlyating rCST9 and rCSTC for evaluation in vivo.

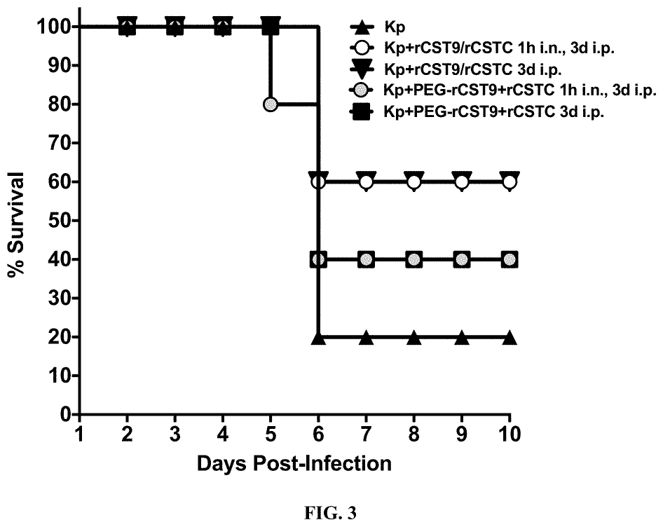

Evaluate lung status following rCST-9 treatment in a mouse model of pneumonia. To evaluate host responses to rCST9/rCSTC, parallel groups of mice (n=4/group) were infected and treated with a combination rCST9/rCSTC (described above) as follows: 1) rCST9/rCSTC was administered i.n. (50 pg of each/mouse) on 1 h PI and i.p (500 pg of each/mouse) on 3d PI, OR 2) a single i.p. dose of rCST9/rCSTC (500 pg of each/mouse) was given on 3d PI. Serum, bronchial alveolar lavage fluid (BALF), lungs, liver and spleen were collected on day 5 PI to analyze bacterial load, lung histology and cytokine profiles. FIG. 3 shows the results from pegylation of rCST9, co-administered with rCSTC, did not improve survival outcomes of NDM-1 Kp infected mice. Balb/c mice (n=15 mice/gp) were i.n. infected with an LD90 challenge with NDM-1 Kp and then treated with an i.n. dose of PEG-rCST9/rCSTC (50 pg of each/mouse) on 1 h PI followed by 500 pg of PEG-rCST9/rCSTC/mouse at 3d PI or mice were administered a single i.p. dose of PEG-rCST9/rCSTC (500 pg of each/mouse) at 3d PI. PEG-rCST9/rCSTC did not improve the survival of infected mice that surpassed rCST9/rCSTC. However, both groups that receive the i.p. administration of rCST9 or PEG-rCST9 in combination with rCSTC on day 3 PI significantly increased survival compared to NDM-1 Kp infected mice (p<0.05). Data are presented as mean±SEM, and asterisk signifies significant differences of p<0.05.

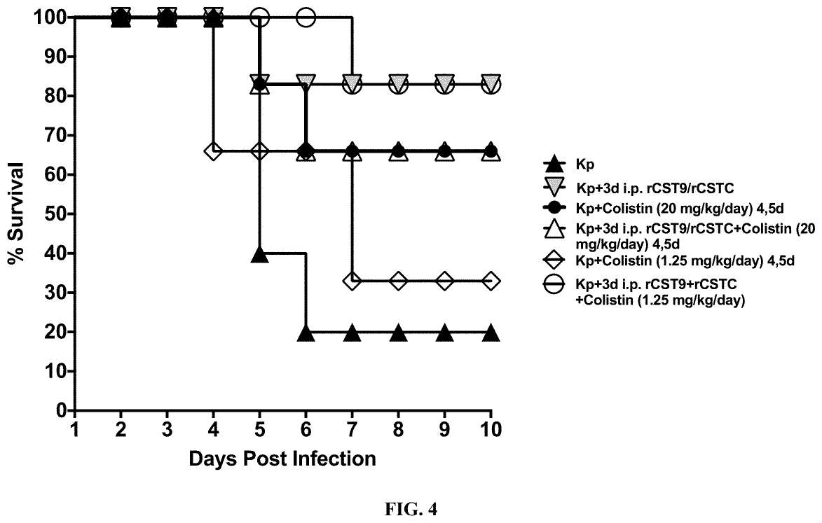

The potential for rCST9/rCSTC treatment to extend the period before successful antibiotic intervention was initiated was determined. For these evaluations, suboptimal doses of colistin were used to determine the extent of protection afforded by rCSTs as well as to minimize side effects/toxicity caused by this antibiotic (n=10 mice/gp). Doses, timing, and route of colistin were chosen based on efficacy studies of colistin in a mouse model of severe, established MDR pneumonia [33] or a very low dose of colistin then adjusted to suboptimal levels for this study. LD90 NDM-1 Kp-challenged mice were treated with the rCST9/rCSTC 3d i.p. treatment and then given 2 doses of colistin at 20 mg/kg/day or 1.25 mg/kg/day on day 4 and 5 PI. Conversely, rCST treatment administered prior to 1.25 mg/kg/day of colistin 4 and 5 days PI significantly improved survival outcomes in NDM-1 Kp-infected mice (FIG. 4; p<0.05). Interestingly, rCST treatment alone confirmed that the rCST treatment led to an unprecedented improvement in survival of NDM-1 Kp-infected mice compared to the low dose of colistin alone and NDM-1 Kp controls (FIG. 4; p<0.05). These results showed that rCST treatment extended the period before antibiotic intervention is initiated and importantly rCST treatment works synergistically with a dramatically lower, less toxic dose of colistin to combat pneumonia.

These data showed that the combination of rCST9 and rCSTC administered to the primary sight of infection (i.n.) prevented lung damage following Kp infection. More, rCST9 and rCSTC given i.p. modulated both systemic Kp as well as lung inflammation resulting in significantly improved survival.

Evaluate alternative dosages of rCST9 and PEG rCST9. The inventors used 1000 pg/mouse of single and combined treatments of rCST9/rCSTC administered at the optimized time(s) 1 h and/or 3d PI as well as 4d and 5d PI using i.n. and/or i.p route(s).

Combined therapies of rCST9 can be evaluated with current antimicrobial agents to minimize ALI/ARDS leading to improved survival (e.g. increase in survival of 10-15%) that surpasses a single therapeutic treatment in an experimental mouse model of bacterial pneumonia, e.g., minimizing ALI/ARDS leading to improved host survival.

Evaluation of combined treatments. Colistin is the one antibiotic NDM-1 Kp 2146 is sensitive to, therefore, the inventors can use the system disclosed hereinabove to evaluate suboptimal dosages of colistin in the two optimized LD90 Kp-rCST9 and rCSTC models. Specifically, colistin is administered at a suboptimal dose on 4d or 5d PI/cystatin treatment. The timing of colistin treatment can also be adjusted to optimize results.

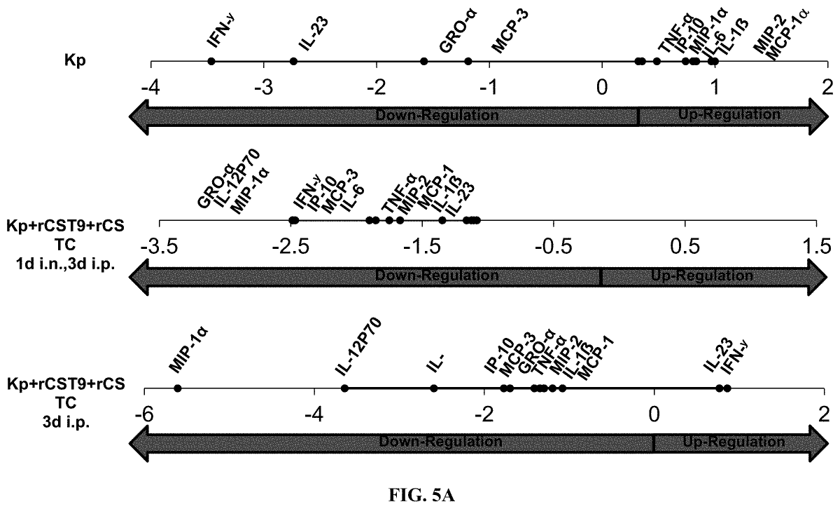

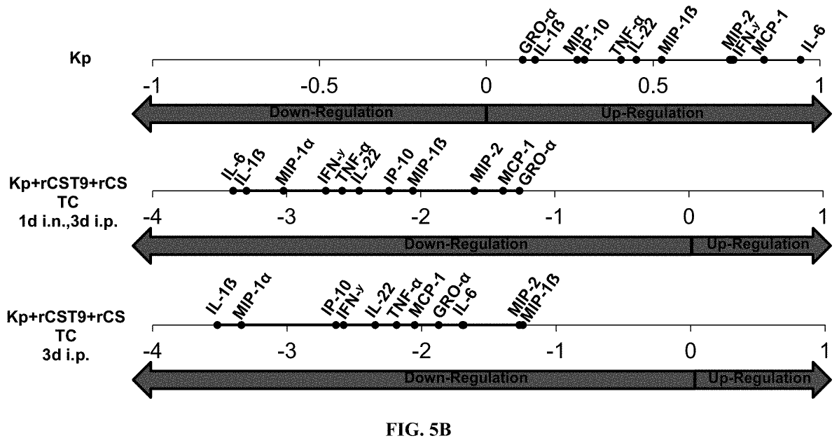

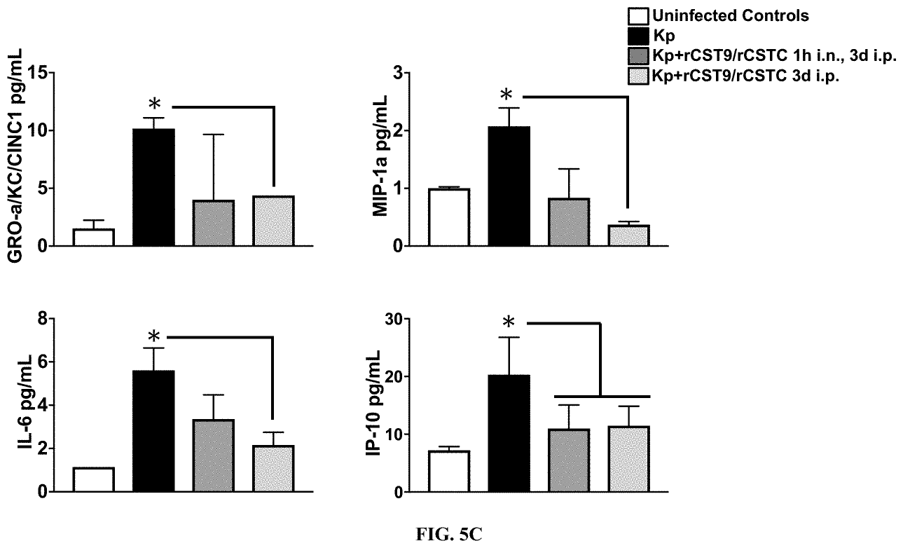

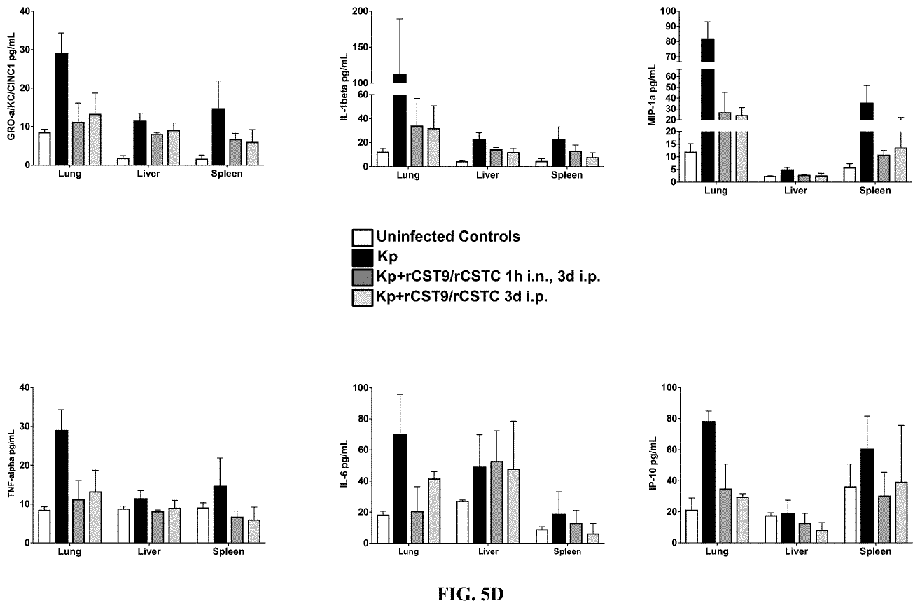

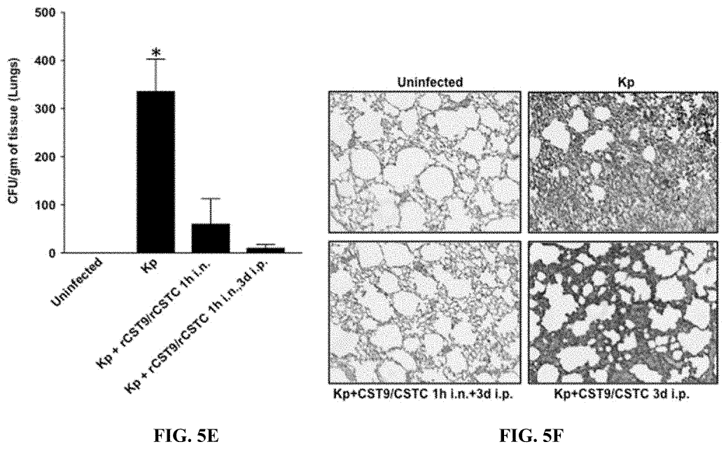

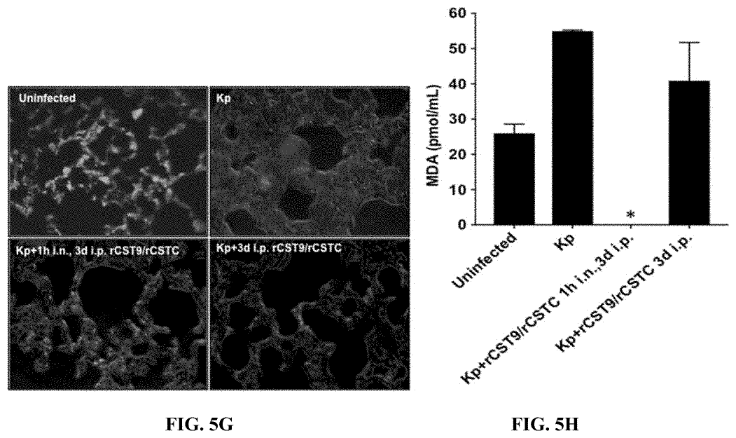

FIG. 5A shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Serum was collected and lungs, livers, and spleens were harvested at 5 days PI. Fold change in the overall cytokine levels in the serum (FIG. 5A) of rCST treated mice were decreased compared to untreated in NDM-1 infected mice.

FIG. 5B shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Serum was collected and lungs, livers, and spleens were harvested at 5 days PI. Fold change in the overall cytokine levels in the lungs (FIG. 5B) of rCST treated mice were decreased compared to untreated in NDM-1 infected mice.

FIG. 5C shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments modulated cytokine secretion in the serum (FIG. 5C).

FIG. 5D shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments modulated cytokine secretion in all tested organs (FIG. 5D).

FIGS. 5E to 5H shows that rCST9/rCSTC treatment modulated inflammatory responses and preserved lung integrity in a mouse model of pneumonia. Balb/c mice (n=6 mice/gp) were i.n. infected with NDM-1 Kp (1.82×10⁸ CFU/mouse) and then treated with an i.n. dose of rCST9/rCSTC (50 pg of both/mouse) at 1 h PI followed by 500 pg of each rCST9/rCSTC/mouse at 3d PI or mice were administered a single i.p. dose of rCST9/rCSTC (500 pg of each/mouse). Both rCST9/rCSTC treatments significantly reduced bacterial burden in the lungs (E). Lung histology (H&E; 40×mag) from the same treated and/or infected mice showed that both rCST treatment regimens minimalized lung pathology caused by NDM-1 Kp (FIG. 5F). rCST treatment reduced apoptotic cells compared to untreated/infected mice (FIG. 5G). MDA detection in the lungs was significantly decreased in rCST-treated and infected mice (FIG. 5H). Data are presented as mean±SEM, and asterisk signifies significant differences of p<0.05.

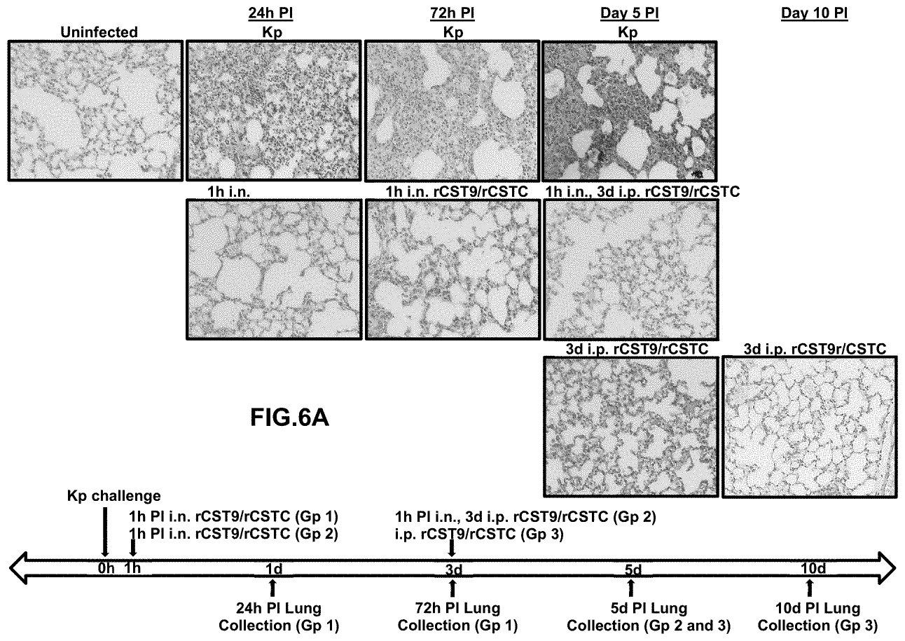

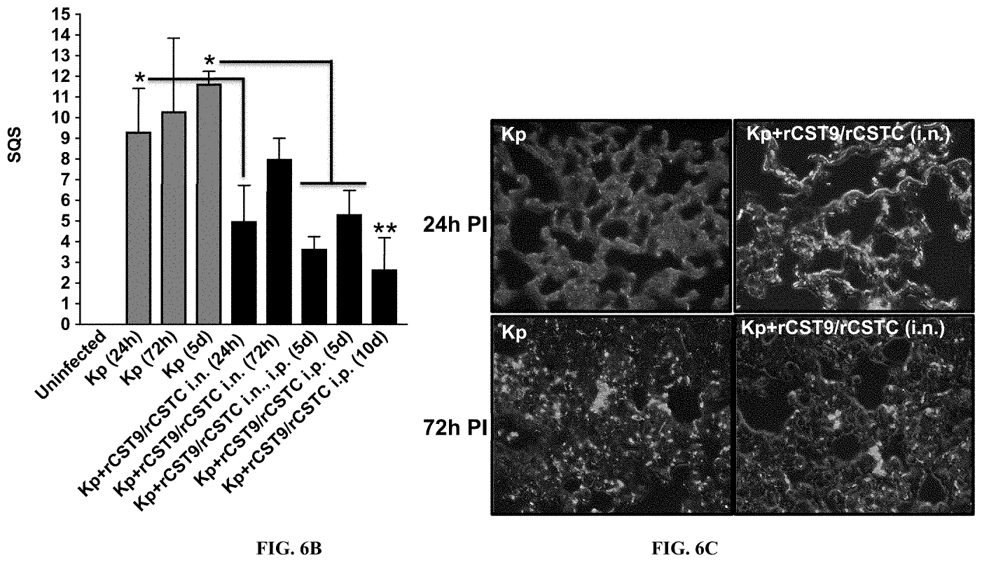

FIGS. 6A to 6C show that rCST treatments preserved lung integrity and prevented long-term lung damage. Balb/c mice (n=4 mice/gp) were i.n. infected with NDM-1 Kp (1.82×10⁸ CFU/mouse) and then treated with an i.n. dose of rCST9/rCSTC (50 pg of each). The lungs were harvested at 24 h and 72 h PI. A parallel group of mice were infected and treated i.n./i.p. or i.p with rCST9/rCSTC as described herein, and then lungs were harvested on 5 and 10d PI. Serial sections of the lung were analyzed for histology (40×mag) and apoptosis by using the TUNEL assay with DAPI to stain cell nuclei. FIG. 6A shows the i.n. administration of rCST9/rCSTC to infected mice markedly diminished immune cell infiltration into the lungs and edema at 24 h and 72 h PI compared to high cellularity and signs of hemorrhaging and edema in the lungs of untreated, infected mice. Further, lungs from our two optimal rCST9/rCSTC treatments on 5d and 10d PI prevented long-term lung damage and showed resolution of inflammation. FIG. 6B shows histolopathological scoring of the lungs (0=no significant changes, 1=slight damage, 2=mild to moderate damage, 3=moderate to severe damage and 4=severe damage in each of the three categories. Results showed that cystain treatments significantly decreased lung damage compared to corresponding time points of infected mice alone. Mice receiving rCSTs at 3d PI and lungs collected from survivors at 5 and 10d PI had mild to no damage compared to infected mice alone groups (*p<0.05 and **p<0.01 respectively). The scoring results were expressed as SQS (mean±SEM). FIG. 6C shows likewise, lungs from the same rCST-treated and infected groups showed markedly fewer apoptotic cells at 24 and 72 h PI. All images are representative of the analysis of 4-6 sections of each mouse.

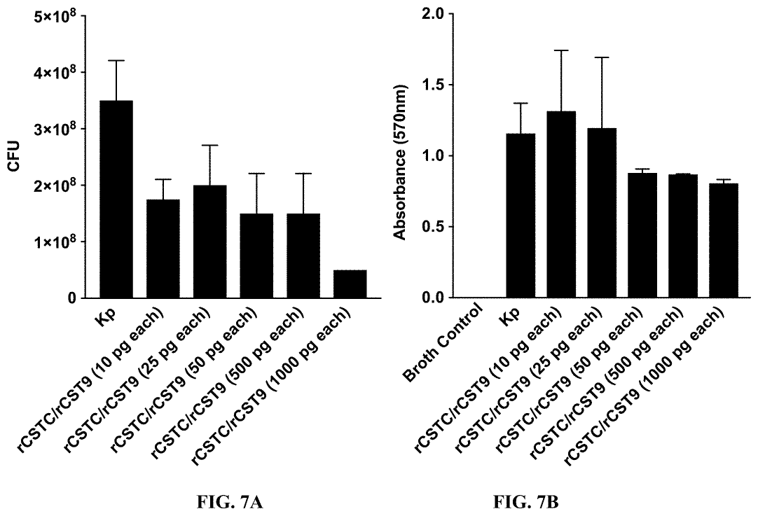

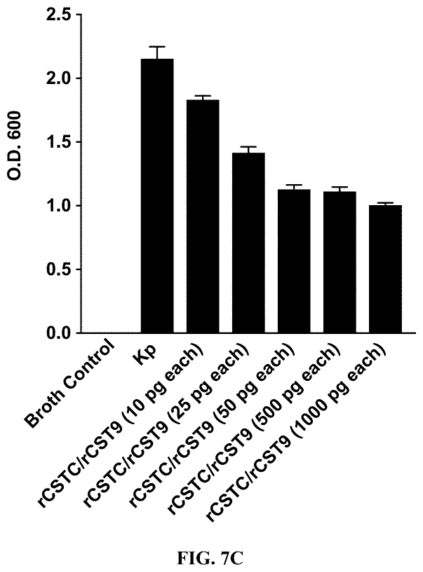

Anti-microbial activity of rCST9/rCSTC against NDM-1 Kp. Past studies also revealed a direct antimicrobial activity of rCSTs. To address this directly, PrestoBlue® was used to determine the viability of rCST9/rCSTC-treated NDM-1 Kp based in the reagents rapid reduction by metabolically active bacteria. These findings revealed that 50, 500, and 1000 pg of rCST9/rCSTC decreased the viability/metabolic activity of 1×10⁶ CFU/mL of NDM-1 Kp during the 6 h incubation compared to untreated NDM-1 Kp and NDM-1 Kp incubated with 10 or 25 pg of rCSTs (FIG. 7A; p<0.05). Further, at 6 h post-incubation, optical density (O.D.) readings and CFUs showed bacterial growth inhibition of NDM-1 Kp incubated with all tested doses of rCSTs compared to NDM-1 Kp alone (FIG. 7B and FIG. 7C; p<0.05). The most substantial decrease in bacterial growth occurred with 50, 500, and 1000 pg of rCSTs compared to lower doses of 10 and 25 pg of rCSTs (FIGS. 7A-C). These results showed that rCST9/rCSTC directly decreased the viability and growth of NDM-1 Kp.

Assess the ability of prophylactic rCST9 to provide a longer window for initiation of antimicrobial treatment leading to prolonged survival (>10-15%) compared to post-treatment alone. As shown above, rCST9's ability to extend the period before antimicrobial treatment was shown against NDM-1 Kp pneumonia.

These findings demonstrate that a single i.n. dose of 50 pg of rCST9 or rCSTC administered 1 h prior to an LD90 dose of i.n. Kp increased survival compared to Kp infected alone [Table 1]. This prophylactic cystatin treatment regime as well as the combination of rCST9/rCSTC followed by colistin 3d or 5d PI to evaluate improvement in survival as well as prolonged survival can also be determined. The prophylactic single or combined cystatin doses should increase survival >15% compared to the LD90 Kp model.

The protective effects of rCST9 against an NDM-1 Kp infection in mice can be translated to a human lung model necessary for translation to drug development in patients. These studies allow for more accurate, clinically relevant assessment of rCST9 to better predict its behavior in patients and will begin to evaluate the PK and potential toxicity of rCST9.

As such, it is possible to evaluate the protective effects of rCST9 against NDM-1 Kp infection in an improved human lung cell model. A new cystatin protein purification protocol using a loose Nickel resin allowing for cleaner purification and milligrams of protein isolation can also be used.

The ability of bacterial organisms to acquire MDR genes is on the rise, making infections caused by MDR pathogens difficult to treat with traditional antibiotics. Therefore, the development of novel, effective therapeutic inventions for these deadly infections is imperative. Herein, it is demonstrated that two human cysteine proteinase inhibitors, known as cystatin 9 and cystatin C, are immunomodulators of inflammation caused by deadly pathogens. This study showed that rCST9 and rCSTC worked synergistically, and in a multi-faceted manner, to modulate excessive, damaging inflammatory responses in pneumonia caused by MDR NDM-1 Kp. Therapeutic efficacy was established using two different rCST9/rCSTC treatment regimens in mice challenged with an LD90 dose of MDR NDM-1 Kp. Post-infection rCST treatment at the primary site of infection (lungs) as well as a single systemic treatment (i.p.) enhanced bacterial clearance and significantly improved survival. Further, it was found that the established rCST9/rCSTC treatment regimens were not toxic or harmful to the host.

Initially, rCSTC was used as a comparative control for rCST9 and then, unexpectedly, discovered that treatment with the combination of rCST9 and rCSTC worked synergistically to combat deadly MDR Kp pneumonia. These efficacy studies determined that low doses and as few as one treatment of rCST9/rCSTC significantly modulated otherwise excessive cytokine secretion to a beneficial level and enhanced bacterial clearance, allowing the host to successfully fight and ultimately resolve the infection, resulting in an unprecedented improvement in survival. Surprisingly, systemic administration of a single i.p. dose of rCST9/rCSTC (500 pg each) after the establishment and systemic dissemination (3d PI) of the infection remarkably improved survival outcomes at levels that were equivalent to the dual i.n./i.p. treatment regimen. Moreover, the single rCST9/rCSTC dose tempered damaging pulmonary and systemic cytokine secretion that decreased lung pathology (e.g. MDA and apoptosis). In fact, in rCST treated mice the lung histology was normal with no signs of long-term damage by 10d PI.

To capitalize on the protective effects of the small, optimal doses of rCSTs, rCST9 was pegylated in an effort to potentially improve its bioavailability in NDM-1 Kp-infected mice. Pegylation is the method of covalently attaching polyethylene glycol to a target, small molecule to improve therapeutic efficacy and pharmacokinetics by enhancing the bioavailability, stability and half-life of small-molecule drugs in vivo [34, 35]. Despite successful pegylation, co-administered PEG-rCST9 and rCSTC actually showed decreased survival outcomes. These results are consistent with testing of higher or more frequent rCST9/rCSTC doses that did not improve survival against NDM-1 Kp pneumonia (data not shown). Additional studies of the PEG-rCST9/rCSTC at lower doses or different combinations will be required to fully appreciate the impact of pegylation. In one non-limiting example, a low-dose, 2 route delivery course was optimal and provided a solid foundation of pharmacokinetic and pharmacodynamic results for immunotherapeutic interventions against pneumonia.

Treatment of pneumonia attributed to NDM-1 Kp remains challenging because of the acquired resistance to most commercially available antibiotics as well as the pathogen's ability to survive for extended periods on environmental surfaces, factors that enhance person-to-person transmission [16, 17, 20, 21, 24]. Combination antibiotic therapy is routinely implemented to treat NDM-1 Kp pneumonia, but these antibiotic poly-treatments are typically accompanied by serious side effects due to the high doses necessary to fight the infection. The NDM-1 Kp strain used in these studies is sensitive to colistin, which is a mixture of polymyxins, specifically polymyxin E, that is considered a last resort treatment for MDR Gram-negative pathogens due to kidney toxicity [36]. Therefore, optimal rCST treatments were accompanied by 20 mg/kg/day or 1.25 mg/kg/day of colistin to determine the extent of protection afforded by rCSTs before antibiotic intervention. Administration of 20 mg/kg/day of colistin 2 times/day for 2d following optimal rCST treatments did not increase survival outcomes that exceeded the single, low dose of rCST alone. However, rCST treatment given prior to the administration of a low, suboptimal dose of 1.25 mg/kg/day of colistin on days 4 and 5 PI significantly improved survival outcomes compared to colistin alone. These results showed that the administration of suboptimal antibiotic therapy allowed enhanced efficacy in the presence of rCSTs that will likely minimize side effects/toxicity caused by the antibiotic, however, additional studies will be required to address this question. It is important to note that neither of the antibiotic doses surpassed protection afforded by the rCST monotherapy.

Finally, these findings showed that 50, 500 and 1000 pg of rCST9/rCSTC directly inhibited NDM-1 Kp metabolic activity and growth. 1000 pg of the rCSTs inhibited NDM-1 Kp metabolic activity and growth nearly equivalent to optimal rCST in vivo doses of 50 and/or 500 pg, the rCST 1000 pg dose did not provide significant protection in the murine model of pneumonia (data not shown). This invention describes the effects of rCST9 [15] and/or rCSTC on deadly MDR bacterial pathogens both in vitro and in vivo for the first time.

Due to the numerous strains of rapidly evolving MDR pathogens, there is an urgent need to develop alternative therapeutic agents to treat these deadly human infections. These findings reveal the multifaceted synergistic, immune-regulatory functions of rCST9/rCSTC. Herein, it is shown that the exogenous co-administration of human rCST9/rCSTC preserved lung integrity, modulated local and systemic cytokine secretion, enhanced anti-bacterial immune responses, and bacterial clearance of MDR NDM-1 Kp pneumonia. These findings showed that a single, low-dose administration of rCST9/rCSTC afforded unprecedented protection without toxic side effects. These findings demonstrate that rCST9/rCSTC provided broad-spectrum protection against pneumonia caused by MDR NDM-1 Kp, and modulated inflammation in multifaceted and unpredicted ways.

Human recombinant CSTC and CST9. Recombinant human Cystatin C was purchased from R&D Systems (Minneapolis, Minn.) and rCST9 was purchased from American Research Products, Inc™ (Waltham, Mass.).

Bacteria preparation. The MDR New Delhi metallo-beta-lactamase-1 (NDM-1) producing Klebsiella pneumoniae BAA-2146™ was purchased from ATCC and expanded for 18 hours in 10 mL of brain heart infusion (BHI) broth while shaking at 37° C. The overnight culture was pelleted by centrifugation and then suspended in PBS. Serial dilutions were performed to obtain the desired concentration. Ten-fold dilutions were plated on BHI agar to confirm experimental dosage.

Pegylation of rCST9. In order to generate an N-terminal mono-PEGylated (PEG) human rCST9 protein, purified rCST9 was incubated with 20 kDa Methoxy PEG Propionaldehyde (M-ALD-20K; Jenkem Technology, Beijing, China) and sodium cyanoborohydride (Sigma-Aldrich, St. Louis, Mo.) at a molar ratio of 1:8:80 in 50 mM sodium acetate buffer (pH 4.5). After 46 hrs incubation at room temperature, the mixture was loaded onto a Superdex 75 column (1.6 cm×60 cm, GE Healthcare, USA) equilibrated with Hank's Balanced Salt Solution (HBSS buffer). Proteins were subsequently eluted and fractionated by using HBSS at a flow rate of 2 mL/min and detected by the absorbance of 280 nm. Fractions containing the PEGylated rCST9 were further identified and protein content profiled by SDS-PAGE analysis.

Mouse model of pneumonia and CST treatments. Eight-week old female Balb/c mice weighing between 21 and 24 grams (Jackson Laboratories) were housed in an Association for Assessment and Accreditation for Laboratory Animal Care (AAALAC)-approved housing facility and permitted to adjust to their environment for 7d prior to procedures, receiving free access to food and water throughout the study. All procedures were approved by the University of Texas Medical Branch IACUC and performed humanely with minimal suffering. We established an LD90 model of pneumonia by anesthetizing mice (n=15-20 mice/gp) with sodium pentobarbital and then challenging them with an i.n. dose of 1.82×108 CFU/mouse of NDM-1 Kp as previously described [15, 42]. One hour PI, mice were administered an i.n. dose of rCST9/rCSTC (50 pg of each) and then, this group of mice was given an i.p. injection of rCST9/rCSTC (500 pg of each) 3d PI. A parallel group of infected mice received only a single i.p. dose of rCST9/rCSTC, PEG-rCST9/rCSTC (500 pg of each), or PEG-rCST9 (500 pg) alone 3d PI. NDM-1-Kp infected mice alone or uninfected mice served as controls. Survival was observed up to 15 days post treatment. Additional groups (n=4 mice/group) of infected and CST-treated mice were euthanized at selected time points (24 h, 72 h, 5 and/or 10d PI) to harvest lungs. Additional groups were treated with i.n. rCSTs (50 pg of each) as described above but they were euthanized at 30 min, 1 h and 3 h to collect lungs for the quantification of lipid peroxidase by-product, known as malondialdehyde (MDA), as described below.

Additional groups of mice (n=15 mice/group) were treated with the optimal CST treatment regimens of an i.n. dose of rCST9/rCSTC (50 pg of each), followed by an i.p. injection of rCST9/rCSTC (500 pg of each) 3d PI or the single i.p. dose of rCST9/rCSTC (500 pg of each). At 4d PI, mice were given 2 separate i.p. injections of colistin (JHP Pharmaceuticals, LLC; colistimethate sodium; 20 mg/kg/mouse or 1.25 mg/kg/day) 8 h apart for 2d. Survival was observed for 15-20 days.

Lung histology, apoptosis, lipid peroxidation, and bacterial burden. Following collection, organs were weighed, then a small portion of the lungs was fixed and processed for hematoxylin and eosin (H&E) staining. A semi-quantitative scoring system was employed to the lung sections collected at 24 h, 72 h, 5d and 10d PI (FIGS. 6A and B). The entire lung section from each condition was analyzed under the following categories: structural abnormalities/congestion, hemorrhaging and cellularity. A lung section from each condition was analyzed in triplicate from 3 individual subjects. Each lung section was given a score ranging from 0-4, whereby 0=no significant changes, 1=slight damage, 2=mild to moderate damage, 3=moderate to severe damage and 4=severe damage in each of the three categories. The semi-quantitative score (SQS) is expressed as the sum of the scores from all three categories. The scoring results were expressed as SQS (mean±SEM).

Apoptotic cells were identified from parallel lung sections by a TUNEL assay using an in situ cell death detection kit (Trevigen) as per the manufacturer's instructions. Nuclei were stained with SlowFade Diamond AntiFade Mountant with DAPI (Invitrogen). Remaining lung materials were homogenized in 1 mL of PBS. Aliquots of lung tissue homogenates were analyzed via a malondialdehyde (MDA) assay kit (Cell Biolabs Inc.), using the manufacturer's instructions, to detect tissue damage induced by oxidative stress. For each lung, 10% (100 ul) of the gravity clarified homogentate was plated on BHI agar to determine the bacterial burden. Bacterial counts were calculated and expressed as CFU/gram of tissue.

Cytokine profile analysis and ELISA kits. Homogenized tissue supernatants and serum (50 ul samples) were analyzed by ProcartaPlex® Mouse Cytokine/Chemokine (Affymetrix) to quantify cytokine production. Samples were processed per the manufacturer's instruction on a Bio-Plex200 instrument (Bio-Rad).

In vitro bacteria viability and growth assay. As a measure of NDM-1 Kp viability, aliquots were treated with Prestoblue® cell viability reagent (Invivogen) following exposure to CSTs. Briefly, 1×106 CFU/mL of NDM-1 Kp were incubated with 10, 25, 50, 500 or 1000 pg of rCST9/rCSTC at 37° C. for 6 hours. Following incubation, 10 L of Prestoblue® reagent was added to each sample and incubated for 1 h before quantification of cell viability via absorbance at 570-600 nm measurement (Bio-Tek; Epoch Model). Parallel aliquots of rCST treated cultures or untreated cultures were used to determine the optical density of the resulting bacterial cultures via spectrophotometry (O.D. 600 nm). Additionally, 100 μL of cultures were plated on BHI plates to quantify colony-forming units (CFU) following an overnight incubation at 37° C. These studies were performed as per a study by Dr. Coban [43] and according to CLSI recommendations.

Statistical Analysis. Where appropriate, results are reported as mean±SEM of two-to-three independent experiments. Analysis of numerical data was determined by one-way ANOVA and Student's t-test using Prism v7.0c software (Graph Pad, San Diego, Calif.). Survival data were analyzed by log-rank analyses with Welch's corrections using Prism software (GraphPad). Differences were considered statistically significant when the p value was <0.05.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Loffek S, Schilling O, Franzke C W. 2011. Series Matrix metalloproteinases in lung health and disease: Biological role of matrix metalloproteinases: a critical balance. Eur Respir J 38:191-208.
2. Zavasnik-Bergant T. 2008. Cystatin protease inhibitors and immune functions. Front Biosci 13:4625-4637.
3. Ochieng J, Chaudhuri G. 2010. Cystatin superfamily. J Health Care Poor Underserved 21:51-70.
4. Bobek L A, Levine M J. 1992. Cystatins—inhibitors of cysteine proteinases. Crit Rev Oral Biol Med 3:307-332.
5. Kopitar-Jerala N. 2006. The role of cystatins in cells of the immune system. FEBS Lett 580:6295-6301.
6. Poteryaeva O N, Falameyeva O V, Korolenko T A, Kaledin V I, Djanayeva S J, Nowicky J W, Sandula J. 2000. Cysteine proteinase inhibitor level in tumor and normal tissues in control and cured mice. Drugs Exp Clin Res 26:301-306.
7. Vray B, Hartmann S, Hoebeke J. 2002. Immunomodulatory properties of cystatins. Cell Mol Life Sci 59:1503-1512.
8. Gauthier S, Kaur G, Mi W, Tizon B, Levy E. 2011. Protective mechanisms by cystatin C in neurodegenerative diseases. Front Biosci (Schol Ed) 3:541-554.
9. Kaeser S, Herzig M, Coomaraswamy J, Kilger E, Selenica M, Winkler D, Staufenbiel M, Levy E, Grubb A, Jucker M. 2007. Cystatin C modulates cerebral-amyloidosis. Nat Genet 39, 1437-1439
10. Kaur G, Levy E. 2012. Cystatin C in Alzheimer's disease. Front Mol Neurosci 5:79.
11. Ervin H, Cox J L. 2005. Late stage inhibition of hematogenous melanoma metastasis by cystatin C over-expression. Cancer Cell Int 5:14.
12. Tian M, Schiemann W P. 2009. Preclinical Efficacy of Cystatin C to Target the Oncogenic Activity of Transforming Growth Factor f3 in Breast Cancer. Transl Oncol 2:174-183.
13. Magister Š, Kos J. 2013. Cystatins in Immune System. J Cancer 4:45-56.
14. Rivera L E, Colon K, Cantres-Rosario Y M, Zenon F M, Melendez L M. 2014. Macrophage derived cystatin B/cathepsin B in HIV replication and neuropathogenesis. Curr HIV Res 12:111-120.
15. Eaves-Pyles T, Patel J, Arigi E, Cong Y, Cao A, Garg N, Dhiman M, Pyles R B, Arulanandam B, Miller A L, Popov V L, Soong L, Carlsen E D, Coletta C, Szabo C, Almeida I C. 2013. Immunomodulatory and antibacterial effects of cystatin 9 against Francisella tularensis. Mol Med 19: 263-275.
16. Khalil M A F, Elgaml A, El-Mowafy M. 2017. Emergence of Multidrug-Resistant New Delhi Metallo-β-Lactamase-1-Producing Klebsiella pneumoniae in Egypt. Microb Drug Resist 23:480-487.
17. Khan A U, Maryam L, Zarrilli R. 2017. Structure, Genetics and Worldwide Spread of New Delhi Metallo-β-lactamase (NDM): a threat to public health. BMC Microbiol 17:101.
18. Halaby T, Reuland A E, Naiemi N, Potron A, Savelkoul P H, Vanenbrouke-Grauls C M, Nordmann P. 2012. A Case of New Delhi Metallo-β-Lactamase 1 (NDM-1)-Producing Klebsiella pneumoniae with Putative Secondary Transmission from the Balkan Region in the Netherlands. Antimicrob Agents Chemother 56:2790-2791.
19. Pesesky M W, Hussain T, Wallace M, Wang B, Andleeb S, Burnham C D, Dantas G. 2015. KPC and NDM-1 Genes in Related Enterobacteriaceae Strains and Plasmids from Pakistan and the United States. J Emerg Infect Dis 21:1034-1037.
20. Barantsevich E P, Churkina I V, Barantsevich N E, Pelkonen J, Schlyakhto E V, Woodford N. 2013. Emergence of Klebsiella pneumoniae producing NDM-1 carbapenemase in Saint Petersburg, Russia, J Antimicrob Chemother 68:1204-6
21. Bosch T, Lutgens S P M, Hermans M H A, Wever P C, Schneeberger P M, Renders N H M, Leenders ACAP, Kluytmans JAJW, Schoffelen A, Notermans D, Witteveen S, Bathoorn E, Schouls L M. 2017. Outbreak of NDM-1-Producing Klebsiella pneumoniae in a Dutch Hospital, with Interspecies Transfer of the Resistance Plasmid and Unexpected Occurrence in Unrelated Health Care Centers. J Clin Microbiol 55:2380-2390.
22. Li J J, Munoz-Price L S, Spychala C N, DePascale D, Doi Y. 2016. New Delhi Metallo-β-Lactamase-1-Producing Klebsiella pneumoniae, Florida, USA. Emerg Infect Dis 22:744-6.
23. Lee C-R, Lee J H, Park K S, Kim Y B, Jeong B C, Lee S H. 2016. Global Dissemination of Carbapenemase-Producing Klebsiella pneumoniae: Epidemiology, Genetic Context, Treatment Options, and Detection Methods. Front Microbiol 7:895.
24. Pitout J D, Nordmann P, Poirel L. 2015. Carbapenemase-Producing Klebsiella pneumoniae, a Key Pathogen Set for Global Nosocomial Dominance. Antimicrob Agents

Chemother 59:5873-84.
25. Hudson C M, Bent Z W, Meagher R J, Williams K P. 2014. Resistance Determinants and Mobile Genetic Elements of an NDM-1-Encoding Klebsiella pneumoniae Strain. PLoS ONE 9: e99209.
26. Robilotti E and Deresinski S. 2014. Carbapenemase-producing Klebsiella pneumoniae. F1000Prime Rep 6: 80.
27. Liang Z, Li L, Wang Y, Chen L, Kong X, Hong Y, Lan L, Zheng M, Guang-Yang C, Liu H, Shen X, Lou C, Li K, Chen K, Jiang H. 2011. Molecular Basis of NDM-1, a New Antibiotic Resistance Determinant. PLOS ONE 6: e23606.
28. Zmarlicka M T, Nailor M D, Nicolau D P. 2015. Impact of the New Delhi metallo-beta-lactamase on beta-lactam antibiotics. Infect Drug Resist 8:297-309.
29. Zhu K, Lu J, Liang Z, Kong X, Ye F, Jin L, Geng H, Chen Y, Zheng M, Jiang H, Li J Q, Luo C. 2013. A quantum mechanics/molecular mechanics study on the hydrolysis mechanism of New Delhi metallo-β-lactamase-1. J Comput Aided Mol Des 27:247-56
30. Bauer T T, Ewig S, Rodloff A C, and Muller E. 2006. Acute respiratory distress syndrome and pneumonia: a comprehensive reivew of clinical data. Clin Infect Dis 43:748-756.
31. Okamoto T, Akuta T, Tamura F, van Der Vliet A, Akaike T. 2004. Molecular mechanism for activation and regulation of matrix metalloproteinases during bacterial infections and respiratory inflammation. Biol Chem 385:997-1006.
32. Hickman-Davis J M, O'Reilly P, Davis I C, Peti-Peterdi, Davis G, Young R, Devlin R B, and Matalon S. 2002. Killing of Klebsiella pneumonia by human alveolar macrophages. Am J Physiol Lung Cell Mol Physiol 282: L944-L956.33.
33. Docobo-Perez F, Nordmann P, Dominguez-Herrera J, Lopez-Rojas R, Smani Y, Poirel L, Pachón J. 2011. Efficacies of colistin and tigecycline in mice with experimental pneumonia due to NDM-1-producing strains of Klebsiella pneumoniae and Escherichia coli. Int J Antimicrob Agents 39:251-4.
34. Mattheolabakis G, Wong C C, Sun Y, Amella C A, Richards R, Constantinides P P, Rigas B. 2014. Pegylation improves the pharmacokinetics and bioavailability of small-molecule drugs hydrolyzable by esterases: a study of phospho-Ibuprofen. J Pharmacol Exp Ther 351:61-635.
35. Harris J M, Chess R B. 2003. Effect of pegylation on pharmaceuticals. Nat Rev Drug Discov 2:214-21.
36. Gurjar M. 2015. Colistin for lung infection: an update. J Intensive Care 3:3.
37. Falagas M E, Kasiakou S K. 2005. Colistin: the revival of polymyxins for the management of multidrug-resistant gram-negative bacterial infections. Clin Infect Dis 40:1333-41.
38. Cimerman N, Kosorok M D, Korant B D, Turk B, Turk V. 1996. Characterization of cystatin C from bovine parotid glands: cysteine proteinase inhibition and antiviral properties. Biol Chem Hoppe Seyler 377:19-23.
39. Bjorck L, Grubb A, Kjellén L. 1990. Cystatin C, a human proteinase inhibitor, blocks replication of herpes simplex virus. J Virol 64:941-3.
40. Kar S, Ukil A, Das P K. 2011. Cystatin cures visceral leishmaniasis by NF-κB-mediated proinflammatory response through co-ordination of TLR/MyD88 signaling with p105-Tp12-ERK pathway. Eur J Immunol 41:116-27.
41. Eaves-Pyles T D, Wong H R, Odoms K, Pyles R B. 2001. Salmonella flagellin-dependent proinflammatory responses are localized to the conserved amino and carboxyl regions of the protein. J Immunol 167:7009-16.
42. Pyles R B, Jezek G E, Eaves-Pyles T D. 2010. Toll-Like Receptor 3 Agonist Protection against Experimental Francisella tularensis Respiratory Tract Infection. Infect Immun 78:1700-1710.
43. Coban A Y. 2012. Rapid determination of methicillin resistance among Staphyloccus aureus clinical isolates by colorimetric methods. J Clin Micro 50: 2191-2193.

What is claimed is:

1. A method of controlling an immune response in a host to a pathogenic bacterial infection comprising:
identifying a subject in need of treatment for infection with a pathogenic bacteria; and
providing a composition comprising recombinant Cystatin 9 (CST9) and a cystatin C (CSTC) provided 3 days apart and in a synergistic amount that is sufficient to restrain or prevent a life-threatening, unrestrained systemic inflammatory response syndrome in a host against a pathogenic bacteria in lungs of the host selected from an acute lung injury, an acute respiratory distress syndrome, or septic shock.

2. The method of claim 1, wherein the systemic inflammatory response syndrome is an acute lung injury, an acute respiratory distress syndrome, or septic shock.

3. The method of claim 1, wherein the CST9 and the CSTC are provided in a synergistic amount.

4. The method of claim 1, wherein the composition is provided concurrently with one or more antibiotics that are bacteriocidal or bacteriostatic against the pathogenic bacteria.

5. The method of claim 1, wherein the composition further comprises one or more antibiotics that are bacteriocidal or bacteriostatic against the pathogenic bacteria.

6. The method of claim 1, wherein the composition is adapted for controlled release over a 4, 6, 8, 12, or 14 hour period.

7. The method of claim 1, wherein the pathogenic bacteria is selected from at least one of: Burkholderia thialandensis, Klebsellia pneumoniae, E. coli O157:H7, Pseudomonas aeruginosa, or Salmonella typhimurium.

8. The method of claim 1, wherein the pathogenic bacteria is multiple drug resistant.

9. The method of claim 1, wherein the pathogenic bacteria is not Francisella tularensis or an obligate intracellular pathogen.

10. The method of claim 1, wherein the pathogenic bacteria is Gram negative.

11. The method of claim 1, wherein the composition is adapted for intraperitoneal, intravenous, parenteral, enteral, pulmonary, intranasal, intramuscular, rectal, or oral administration.

12. The method of claim 1, wherein both Cystatin 9 (CST9) and Cystatin C (CSTC) are provided intranasally when the systemic inflammatory response syndrome is an acute lung injury, an acute respiratory distress syndrome, or both.

13. The method of claim 1, wherein at least one of the Cystatin 9 (CST9) or Cystatin C (CSTC) are provided in an amount of 1-500 picograms/kilo.

14. The method of claim 1, wherein at least one of the Cystatin 9 (CST9) or Cystatin C (CSTC) is PEGylated.

15. The method of claim 1, further comprising at least one of: a synergistic amount of a polymyxin antibiotic; a sub-optimal dose of a polymyxin antibiotic, wherein the dose is not neurotoxic, nephrotoxic, or both; or a synergistic amount of colistin.

16. A method of controlling an immune response in a host to a pathogenic bacterial infection comprising:
identifying a subject in need of treatment for infection with a pathogenic bacteria; and
administering a composition comprising recombinant Cystatin 9 (CST9) and a cystatin C (CSTC), in an amount sufficient to restrain or prevent a life-threatening, unrestrained systemic inflammatory response syndrome in a host against a pathogenic bacteria.

17. The method of claim 16, wherein the step of administering the recombinant Cystatin 9 (CST9), a cystatin C (CSTC), or both CST9 and CSTC is at least one of: after onset of symptoms, at least three days post-infection, or at least three days post-exposure.

18. The method of claim 16, further comprising at least one of: a synergistic amount of a polymyxin antibiotic; a sub-optimal dose of a polymyxin antibiotic, wherein the dose is not neurotoxic, nephrotoxic, or both; or a synergistic amount of colistin.

19. A composition comprising:
a recombinant Cystatin 9 (CST9) and Cystatin C (CSTC) formulated for intranasal or parenteral administration and in a synergistic amount sufficient to restrain a life-threatening, unrestrained systemic inflammatory response syndrome in a host against a pathogenic bacteria in lungs of a host selected from an acute lung injury, an acute respiratory distress syndrome, or septic shock.
